(19) United States
(12) Patent Application Publication
(10) Pub. No.: US 2022/0017870 A1
(43) Pub. Date: Jan. 20, 2022

(54) METHODS OF IDENTIFYING THERAPEUTIC TARGETS FOR TREATING ANGIOGENESIS

(71) Applicant: Advanced Solutions Life Sciences, LLC, Louisville, KY (US)

(72) Inventors: James B. Hoying, Manchester, NH (US); Hannah A. Strobel, Manchester, NH (US); Sarah M. Moss, Manchester, NH (US)

(21) Appl. No.: 17/375,742

(22) Filed: Jul. 14, 2021

Related U.S. Application Data
(60) Provisional application No. 63/051,957, filed on Jul. 15, 2020.

(51) Int. Cl. C12N 5/071; C12N 5/00; C12N 5/0786; G01N 33/50; C12N 5/0775

(52) U.S. Cl. CPC C12N 5/0691; C12N 5/0037; C12N 5/0645; C12N 2500/84; C12N 5/0668; C12N 2513/00; C12N 2533/54; G01N 33/5044

(57) ABSTRACT

Provided herein is a method for assessing angiogenic effects of a test composition, the method including: providing human microvessel (MV) fragments selected to correspond to a desired patient profile; embedding the human MV fragments in a gel matrix of a three dimensional (3D) in vitro culture; providing serum free media to the 3D in vitro culture; contacting the 3D in vitro culture comprising embedded human MV fragments with a test composition; and assessing the angiogenic effects of the test composition by measuring at least one angiogenic growth parameter of the 3D in vitro culture comprising embedded human MV fragments. Also provided herein are 3D in vitro cultures useful in the disclosed methods.

### RELATED APPLICATIONS

This application claims priority to U.S. Provisional Patent Application 63/051,957, filed Jul. 15, 2020, the content of which is hereby incorporated by reference in its entirety.

### TECHNICAL FIELD

The present disclosure relates to field of 3D angiogenesis models and their methods of use.

### BACKGROUND

Angiogenesis, the growth of new blood vessels, is a fundamental biological process essential for human health. Dysregulation of angiogenesis is associated with many pathological conditions, including cancer, diabetes, immunological disorders, and more. The key to treating many of these devastating diseases may be undiscovered drug targets that regulate angiogenic activity. A critically important factor in identifying a viable drug target is the incorporation of physiological relevance in the assay design as early as possible in the discovery process. Existing endothelial cell-based models have not captured the considerable complexity of native angiogenesis, a highly dynamic and multi-cell type process.

A microvascular system, Angiomics™, has been developed involving the isolation and culture of intact microvessel fragments from human adipose tissue. These microvessels, when embedded in a 3D matrix environment, sprout, grow, and form an in vitro, stable neovascular network in a manner similar to native angiogenesis. This microvessel system captures more of the complexity of native angiogenesis than existing in vitro angiogenesis models, which often only contain only one or two cell types in a 2D environment. The system has demonstrated flexibility and utility of the model as a tool for studying biological mechanisms of angiogenesis and therapeutic potential.

A need exists for improved models and methods that approximate the complexity of native angiogenesis and may be used to identify new drug targets for the treatment of angiogenesis.

### SUMMARY

The following summary is provided to facilitate an understanding of some of the innovative features unique to the present disclosure and is not intended to be a full description. A full appreciation of the various aspects of the disclosure can be gained by taking the entire specification, claims, drawings, and abstract as a whole.

Provided herein are 3D angiogenesis models and their methods of use in identifying and validating drug targets.

In some aspects, the present disclosure concerns methods for assessing or measuring angiogenic effects. In some aspects, the methods include providing human microvessel fragments selected to correspond to a desired patient profile and embedding the human microvessel fragments in a gel matrix of a three dimensional (3D) in vitro culture. In other aspects, the methods also include providing a serum free media to the 3D in vitro culture with the embedded human microvessel fragments and then contacting the 3D in vitro culture with a test composition. In additional aspects, the methods can also include then assessing the angiogenic effects of the test composition by measuring at least one angiogenic growth parameter of the 3D in vitro culture.

In some aspects, the desired patient profile may be selection based on a shared underlying condition or trait. In other aspects, the desired patient profile may be of a heterogeneous selection of patients.

In some aspects, the gel matrix includes collagen. In further aspects, the gel matrix may include fibrin and/or Matrigel. In some aspects, the gel matrix may include collagen and fibrin blends and/or collagen and Matrigel blends. In other aspects, the serum free media is selected for low angiogenic growth conditions or for medium angiogenic growth conditions or for high angiogenic growth conditions.

In some aspects, angiogenic growth is quantified directly by measuring vessel length density of parent microvessels and neovessels to determine a neovessel:parent microvessel ratio. In other aspects, angiogenic growth is quantified indirectly by Alamar Blue assay performed on the 3D in vitro culture. In further aspects, angiogenic growth is quantified indirectly by MMP-14 assay performed on a lysate of the 3D in vitro culture. In other aspects, angiogenic growth is quantified by lectin staining and/or computational analysis or measurement.

In further aspects, the methods may also include suspending a permeable transwell over the gel matrix. In some aspects, the permeable transwell comprises additional cells. In further aspects, the serum free media covers the additional cells in the permeable transwell. In certain aspects, the additional cells include macrophages. In further aspects, the additional cells are autologous to the human microvessels. In some aspects, the permeable transwell also includes the gel matrix.

In some aspects of the methods, the test composition is determined to inhibit angiogenesis when quantified neovessel growth in the 3D in vitro culture contacted with the test composition is lower than the control value. In other aspects, the test composition is determined to promote angiogenesis when quantified neovessel growth in the 3D in vitro culture contacted with the test composition is higher than the control value. In additional aspects, the methods may also include comparing the angiogenic effects of the test composition of the 3D in vitro culture with angiogenic effects in a further 3D in vitro culture contacted with at least one compound known to influence angiogenesis.

In some aspects, the present disclosure concerns a three-dimensional (3D) in vitro culture that includes a gel matrix, a permeable transwell and a basal cell medium. In some aspects, the gel matrix includes a first tissue extract and collagen. In other aspects, the permeable transwell includes a second tissue extract. In some aspects, the basal cell medium is a serum free medium. In some aspects, the permeable transwell is suspended over the gel matrix and the basal cell medium covers the second tissue extract.

In other aspects, the first tissue extract and the second tissue extract are both selected from human microvessels; macrophages, and mesenchymal stem cells. In further aspects, the first tissue extract and the second tissue extract are not identical. In additional aspects, the first tissue extract and the second tissue extract are autologous.

In one aspect, the present disclosure concerns a method for determining whether a test compound modulates angiogenesis, the method including: providing a 3D angiogenesis model with intact native parent microvessels embedded in a gel matrix; contacting the 3D angiogenesis model with a test composition or compound; subjecting the 3D angiogenesis model to dynamic conditions, whereby parent microvessels are stimulated to sprout neovessels; quantifying neovessel growth in the 3D angiogenesis model; comparing the neovessels growth in the 3D angiogenesis model with a control value; and determining that the test compound modulates angiogenesis when the quantified neovessel growth in the 3D angiogenesis model contacted with the test compound is higher or lower than the control value. In other aspects, the test compound or composition inhibits neovessel growth as compared to a control value.

In another aspect, a method for validating a potential target for influencing angiogenesis is provided, the method including: providing a 3D angiogenesis model with intact native parent microvessels embedded in a gel matrix; contacting the 3D angiogenesis model with a compound known to modulate the potential target; subjecting the 3D angiogenesis model to dynamic conditions, whereby parent microvessels are stimulated to sprout neovessels; quantifying neovessel growth in the 3D angiogenesis model; comparing the neovessel growth in the 3D angiogenesis model with a control value; and determining that the potential target influences angiogenesis when the quantified neovessel growth in the 3D angiogenesis model contacted with the compound known to modulate the potential drug target is higher or lower than the control value.

In another aspect, a method for identifying a gene target for angiogenesis is provided, the method including: providing a 3D angiogenesis model with intact native parent microvessels embedded in a gel matrix; contacting the 3D angiogenesis model with at least one compound known to influence angiogenesis; subjecting the 3D angiogenesis model to dynamic conditions, whereby parent microvessels are stimulated to sprout neovessels; determining an RNA expression profile of the 3D angiogenesis model contacted with the at least one compound known to influence angiogenesis; comparing the RNA expression profile of the 3D angiogenesis model contacted with the at least one compound known to influence angiogenesis with a control RNA expression profile; and identifying a gene as a target for angiogenesis when RNA expression for the gene in the 3D angiogenesis model contacted with the compound known to influence angiogenesis is higher or lower than RNA expression for the same gene in the control RNA expression profile.

### BRIEF DESCRIPTION OF THE DRAWINGS

The aspects set forth in the drawings are illustrative and exemplary in nature and not intended to limit the subject matter defined by the claims. The following detailed description of the illustrative aspects can be understood when read in conjunction with the following drawings, where like structure is indicated with like reference numerals and in which:

FIG. 1. Phase images of a human MV isolate (open arrows+MV) and 3D matrix culture undergoing angiogenesis (arrows=neovessels, *=parent MVs). Isolated MVs are intact and comprised of numerous cell types thought important in microvessel stability and angiogenesis (% determined by flow cytometry of dissociated, isolated microvessels).

FIG. 2. Two example results of a phenotypic screen of epigenetic drugs on angiogenesis. Serum-free, 3D cultures of microvessels were treated with NCE1 and NCE2 (10 μM) and relative total vessel length assessed over time (y-axis).

FIG. 3. A) Phase contrast image of parent microvessel and neovessel sprout. MMP-14 stain of a B) parent and C) neovessel. D) ELISA showing MMP-14 expression in MV cultures (MVC). The rhMMP-14 group is of known MMP14 amounts calibrated the ELISA.

FIG. 4. Heat map comparing gene expression between MV with high and low angiogenic potential. Each lot of MV (horizontal row) was scored on a scale of 0-5, with 0 indicating MV death, 1 indicating no growth, 3 indicating average growth, and 5 indicating excessive growth.

FIG. 5. Effect of DYRK3 inhibition on MV growth. Phase contrast images of MVs after 9 days of culture, with a single 3-hour exposure of DYRK3 inhibitor after initial neovessel sprouting (day 4). Fewer neovessels are apparent in the treated sample (right panel). Scale=100 μm. Arrows point to neovessels.

FIG. 6. Diagram of the “core in field” format for collecting angiogenic neovessels (A). An example measurement between mature and angiogenic vessels (B). Arrow=matrix interface.

FIG. 7. A transwell insert containing cells is placed above a microvessel culture in a well plate. After flooding with medium, signaling molecules produced by the cells may affect microvessel growth.

FIG. 8. Microvessels can be placed in a transwell insert to evaluate the effects of microvessels on cellular behavior.

FIG. 9. Microvessels cultured alone rarely cross the tissue interface. When mixed with macrophages (MP), they readily cross the interface. However, medium conditioned by macrophages using a transwell insert (MP cond) does not increase crossing.

FIG. 10. Microvessel conditioned medium increases macrophage crossing events.

### DETAILED DESCRIPTION

The following description of particular aspect(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes or compositions are described as an order of individual steps or using specific materials, it is appreciated that steps or materials may be interchangeable such that the description of the invention may include multiple parts or steps arranged in many ways as is readily appreciated by one of skill in the art.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third,” etc., may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Wild, D., The Immunoassay Handbook, 3rd Ed., Elsevier Science, 2005; Gosling, J. P., Immunoassays: A Practical Approach, Practical Approach Series, Oxford University Press, 2005; Antibody Engineering, Kontermann, R. and Dübel, S. (Eds.), Springer, 2001; Harlow, E. and Lane, D., Antibodies: A Laboratory Manual, Cold Spring Harbor Laboratory Press, 1988; Ausubel, F. et al., (Eds.), Short Protocols in Molecular Biology, Wiley, 2002; J. D. Pound (Ed.) Immunochemical Protocols, Methods in Molecular Biology, Humana Press; 2nd ed., 1998; B. K. C. Lo (Ed.), Antibody Engineering: Methods and Protocols, Methods in Molecular Biology, Humana Press, 2003; and Kohler, G. and Milstein, C., Nature, 256:495-497 (1975); the contents of each of which are incorporated herein by reference.

Angiogenesis, or the sprouting of new blood vessels from existing blood vessels, plays a key role in a plethora of medical conditions. It is essential for tissue health and processes such as wound healing and implant engraftment. Insufficient angiogenesis can lead to tissue death, compromised wound healing, and graft failure. Undesired or excessive angiogenesis can be equally problematic. This pathology is a hallmark of cancerous tumors, which affect more than 1 million people each year, and certain types of retinopathies. Additionally, as regenerative medicine industries continue to emerge, there remains a critical emphasis on tissue vascularization, an intrinsically angiogenesis-dependent process. Importantly, microvessels and the associated angiogenic process are complex “tissues” comprised of numerous cell types organized in a discreet structure. As such, there are considerable opportunities to modulate microvessel biology, and specifically angiogenesis, beyond endothelial cell-targeting growth factors.

In identifying potential drug targets, the more informative a screen is, the more efficient the screening process. This is particularly relevant and challenging in dynamic biological systems such as angiogenesis. To date, available angiogenesis assays involve either reductionist endothelial cell cultures or animals. Ideally, an assay would recapitulate as much of the native angiogenesis dynamics as possible, while providing multiple functional readouts and remaining simple to use and cost-effective. Even more ideal would be an assay or screen that provides functional readouts for a desired potential patient profile or population subset to further identify targets particular to that population subset.

The 3D in vitro culture model of the present disclosure includes intact, true microvessel fragments that retain the structure, cellular complexity, and phenotypic plasticity of native blood microvessels. When embedded in a tissue matrix, neovessels will sprout from the parent microvessel, grow, inosculate with each other, and form a neovascular network analogous to native angiogenesis. The systems provided herein serve as an invaluable tool to study the biology of angiogenesis. Here, the MV system is incorporated into a robust, throughput, angiogenesis assay with multiple quantitative functional readouts. The disclosed system is useful for the identification of multiple potentially novel drug targets, representing a variety of protein classes, related to microvascular biology and angiogenesis.

In some aspects, the present disclosure concerns the application of a test agent to a three-dimensional angiogenesis model. Provided herein is a 3D in vitro angiogenesis model that meets the desired requirements of native angiogenesis dynamics and allows rapid identification of new drug targets. In some aspects, the 3D model is established through the introduction of microvessel fragments in a 3D matrix. Intact isolated microvessel fragments (MVs) that are obtained from living subjects retain their native structure and multi-cellular composition when cultured in a 3D matrix and will undergo sprouting angiogenesis similarly to vessels in the native, in vivo environment. The application of an MV system into an informative in vitro angiogenesis assay compatible with existing assessment approaches (e.g. high content analysis) provides a more biologically relevant assay to identify and qualify potential, novel angiogenic targets. In some aspects, a 3D in vitro human angiogenesis drug-target discovery model is provided. Isolated native microvessels undergo angiogenic sprouting and growth when embedded in a 3D matrix. It is key for an assay to define endpoints with physiological relevance. In some aspects, the defined endpoint comprises neovessel growth (angiogenesis). The 3D angiogenesis model is configured for optimal media/culture conditions, thereby enabling the simultaneous identification of pro- or anti-angiogenesis drug targets, optionally in a 96 well plate format.

In some aspects, the present disclosure relates to an informative, in vitro 3D model of angiogenesis for identifying new potential gene targets for treating (e.g., promoting or inhibiting) pathological angiogenesis. In some aspects, the present disclosure provides for a MV culture of a desired patient profile in the 3D in vitro model. In some aspects, the cells from the 3D culture can be assayed for varying levels of gene expression, enzymatic activity, and similar to assess for angiogenic effects, as well as through visualization and measurement of angiogenesis. Such additional steps are known and may include polymerase chain reactions, RNA isolation, DNA isolation, western blotting, Southern blotting, northern blotting, HPLC-MS/MS, MALDI-TOF, phenotypic screening, nucleic acid and/or protein sequencing, kinase assays, ELISA, electrophoresis, chromatography, flow cytometry and the like. In some aspects, visualization can be achieved through antibody and/or fluorophore labeling. In certain aspects, proteins and/or genes may present as markers of an agent's effect on angiogenesis. For example, as set forth herein, the protein DYRK3 is provided as an example of use of a readout protein for drug testing.

In some aspects, the present disclosure concerns methods for assessing, measuring or determining the angiogenic effects of an agent or a composition of interest. For example, it can be a great benefit to determine if a composition being developed possesses any angiogenic response, either inhibitory or stimulatory. As identified above, angiogenesis can occur in many conditions and depending on the condition, it may be of benefit to promote angiogenesis or stimulate angiogenesis. In other conditions, it may be more of a benefit to inhibit angiogenesis. Accordingly, by providing the methods of the present disclosure as set forth herein, determination of the potential effects on angiogenesis of a composition of interest or a test composition is provided.

In some aspects, it can be of further benefit to determine the potential angiogenic effects a composition may have on a particular population or profile of a population. It is understood that many different genes, proteins, growth factors, enzymes, and overall signaling mechanisms contribute to angiogenesis and as such, many factors may contribute to how an individual may respond to a pro- or anti-angiogenic compound, including genetics, lifestyle, diet, gender, medications, age, weight, underlying condition or disease(s), air quality, occupation, or other events such as recent injury or surgery. In some aspects, it may therefore be beneficial to cull or select the MVs that will be incorporated into the 3D culture from a desired patient population profile that represents one or more shared characteristics that can be representative a potential class of patients. For example, if one skilled in the art wants to assess how a test compound may perform in cancer patients, MVs from patients with varying types of cancer may be collected and cultured. If one skilled in the art prefers to analyze angiogenesis as it relates to a specific cancer type, the selected MVs can be obtained from patients having that specific cancer. Similarly, if one skilled in the art prefers to observe how a test compound may perform across a broad cross-section of the population, the selected MVs may be obtained from a heterogeneous collection of patients or people. In some aspects, a profile may refer to one or more particular characteristics or traits that are shared or present in the selected patient population, such as a shared genetic condition, lifestyle, and/or disease.

In some aspects, the present disclosure concerns methods for assessing the angiogenic effects of a test composition by providing the MV fragments from the desired patient population to a gel matrix of a 3D in vitro culture. In some aspects, the MV fragments can be provided to the gel matrix by embedding the MV fragments in the gel. In some aspects, the microvessels are provided to the gel matrix by suspending the MV fragments in a pre-polymerization solution, such as a collagen solution, a fibrin solution, a Matrigel solution, or combinations thereof, and then permitting and/or initiating polymerization to form the gel.

In some aspects, the present disclosure concerns providing MV fragments to a 3D in vitro culture. In certain aspects, the MV fragments are obtained from the tissue(s) of a patient with the desired profile for analysis. In some aspects, the MV fragments are obtained from digesting tissue from the patient with an enzyme. In some aspects, the tissue is contacted with a collagen to break up or disrupt the tissue. In some aspects, the tissue may be pre-treated to allow the MVs to be released or loosened within the tissue so that the MVs may have ready access to the test-composition and improved space or freedom to respond to the test composition.

In some aspects, the present disclosure concerns providing the MV fragments to the gel matrix by suspending or overlaying the MV fragments in a solution above the gel.

In certain aspects, the MV fragments are provided to the gel with other cell types. In some aspects, the 3D in vitro culture may include one or more additional cell types or secondary tissues other than the MVs. Such additional cell types may be useful to approximate a particular tissue environment. For example, in some aspects, the 3D in vitro culture may further include parenchymal and/or stromal cell types. In certain aspects, the 3D in vitro culture may further include one or more of tumor cells, tumor spheroids, tumor organoids, and the like. Optionally, such cells may be autologous cells obtained from the same patient who provided parent native microvessels. Optionally, such cells may be nonautologous, or a mixture of autologous and nonautologous cells. For example, the MV fragments can be co-cultured with one or more of macrophage cells, progenitor endothelial cells, mesenchymal stem cells, fat cells, mesodermal cells, hematopoietic cells, parenchymal cells, stromal cells, muscle cells, neuronal cells, dermal cells, tumor cells, tumor spheroids, tumor organoids, and the like.

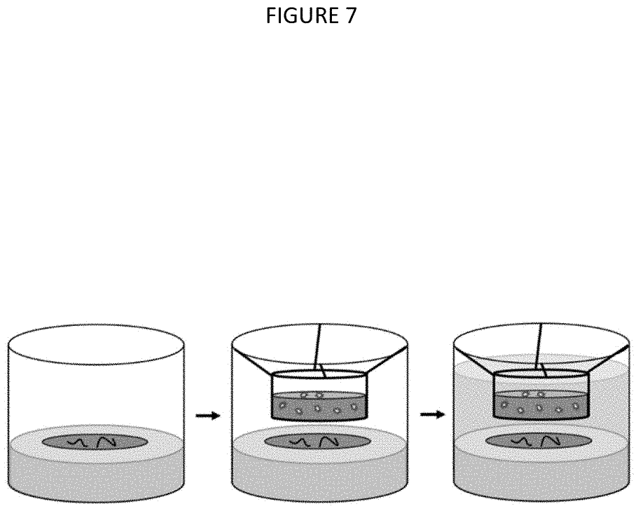

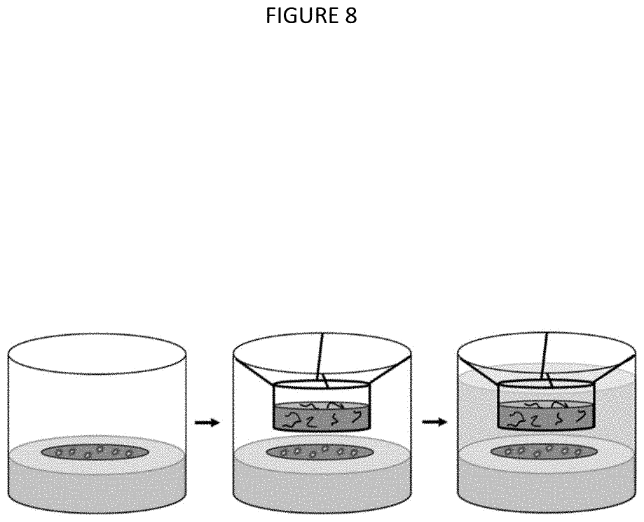

In some aspects, the MV fragments can be provided to the gel in proximity or in a shared solution with other cells types. For example, FIGS. 7 and 8 set forth an exemplary arrangement wherein permeable transwell inserts are placed above the gel matrix and the collective well or plate is filled with sufficient medium to over the gel and the insert placed above. Accordingly, the cells in the insert are in fluid communication with the cells in the underlying gel matrix. The cells in the insert may be MV fragments or may be other cells types, such as macrophages, mesenchymal stem cells, and/or others. The cells in the matrix can be of the same or a differing cell type. The insert may be of a 2D in vitro culture or of a further 3D gel matrix. Optionally, the additional cell types, such as parenchymal cells, stromal cells, tumor cells, tumor spheroids, tumor organoids, and the like, may be disposed in the 3D angiogenesis model such that the cells are at least partially physically sequestered from the microvessels, such that the cells do not unduly interfere with analysis of microvessel angiogenesis using the techniques described herein.

In some aspects, a serum free media is provided to the 3D in vitro culture. In some aspects, the serum free medium can be selected to provide low, medium, or high angiogenic conditions. The growth rate itself can be relative to the sample size being calculated. For example, phase contrast viewing of the MVs provides a field of view or sampling area with few MVs. In such aspects, a low rate can be considered to be of about 0 to about 1 mm/mm², including about 0.1, 0.2, 0.3, 0.4, 0.5, 0.6, 0.7, 0.8, and 0.9 mm/mm². In such aspects, medium growth can be considered from about 1 to about 2 mm/mm², including about 1.1, 1.2, 1.3, 1.4, 1.5, 1.6, 1.7, 1.8, and 1.9 mm/mm². In such aspects, high growth can be of about 2 mm/mm² or more. In other aspects, the sampling area may be from a different modality and include larger numbers of MVs, such as with confocal microscopy. In such aspects, low growth can be of about 10 mm/mm² or less, medium growth of from about 10 mm/mm² to about 25 mm/mm² and high growth above about 25 mm/mm². As set forth in the working examples, one aspect of the present disclosure concerns the identification that angiogenesis can proceed at varying rates depending on the type or content of the serum free medium provided to the MV fragments in the 3D in vitro culture. Table 1 identifies several medium bases and supplements thereto that can affect the rate of angiogenic growth. As set forth in Table 1, the base media included RPMI (Roswell Park Memorial Institute), MCBD (molecular cellular and developmental biology), William's E, DMEM/F12 (Dulbecco's Modified Eagle Medium), and CMRL (Connaught Medical Research Laboratories). Other media, such as MEM (minimal essential medium), Milieux 199, Ham's, McCoy's, IMDM (Iscove's Modified Dulbecco's Medium), DMEM, EMEM (Eagle's Minimum Essential Medium), F-10 and F-12, including combinations thereof are also contemplated. In some aspects, the basal medium may include one or more of L-glutamine, biotin, B12, PABA, amino acids, vitamins, sodium pyruvate, glucose, HEPES, glycine, serine, balanced salt, nonessential amino acids, sodium pyruvate, ferric nitrate, no/few hormones, no/few growth factors, and/or no/few trace elements, In some aspects, media including CMRL and MCBD allow for a low to medium rate of angiogenic growth when provided to the MVs in 3D in vitro culture. In other aspects, media including DMEM/F12, William's E, and RPMI provide medium to high amounts of angiogenic growth.

TABLE 1

| BASE MEDIUM | SUPPLEMENT | OUTCOME |
|---|---|---|
| DMEM/F12 | Sato + VEGF | 3 |
| DMEM/F12 | B27 | 3 |
| DMEM/F12 | FBS | 2 |
| William’s E | Sato + VEGF | 4 |
| William’s E | Hepatocyte | 2-4 |
| | Maintenance | |
| RPMI | None | 3 |
| RPMI | B27 | 4 |
| RPMI | B27 + VEGF | 5 |
| RPMI | FBS | 4 |
| CMRL | B27 | 1 |
| CMRL | Islet Supplement | 0 |
| | Cocktail | |
| MCBD | Islet Supplement | 2 |
| | Cocktail | |
| MCBD | B27 | 3 |

As further set forth in Table 1, supplements such as Sato supplement (e.g., ˜10 mg/mL BSA (bovine serum albumin), ˜10 mg/mL transferrin, ˜1.6 mg/mL putrescine, ˜6 μg/mL progesterone, ˜4 μg/mL sodium selenite), VEGF (vascular endothelial growth factor), B27 supplement, hepatocyte maintenance supplement, islet supplement cocktail, and FBS (fetal bovine serum) can be added to further regulate angiogenic growth. As also identified in Table 1, supplements such as Sato, VEGF, and B27 may increase angiogenic growth in some serum-free media. In other aspects, supplements such as FBS may in some instances have lower or indifferent effects on angiogenic growth. In other aspects, additional supplements may also be included to yield an effect on angiogenic growth. As demonstrated in Table 1, the addition of serum to the serum free medium can reduce angiogenic growth.

As also identified herein, the serum free medium can cover the base gel matrix. In further aspects, the serum-free medium can cover both the gel matrix and a suspended or overlaid transwell insert to provide a fluid medium connection for signals or excreted factors between the insert and the underlying gel matrix.

In further aspects, different amounts of the supplements can be utilized with the different media. For example, Table 2 sets forth a demonstration of utilizing different media with differing amounts of supplements.

TABLE 2

| Base Medium | Supplements |
|---|---|
| RPMI | B27 |
| RPMI | B27 + 50 |
| | ng/mL VEGF |
| RPMI | B27 + 10 |
| | ng/mL VEGF |
| DMEM/F12 | Sato + 50 |
| | ng/mL VEGF |
| DMEM/F12 | Sato + 10 |
| | ng/mL VEGF |
| CMRL | B27 |

In some aspects of the present disclosure, the test agent is administered or applied or contacted with the gel matrix. Such administration can be accomplished through administering directly into the gel matrix and/or transwell insert, or by administration to the serum-free medium. In some aspects, a user may prefer to include a period of time between establishing the 3D in vitro cell culture and administering a test composition thereto. Such a period of time may be dependent on conditions preferred for the MV fragments prior to application of the test composition. For example, in some aspects, it may be desirable to test the MV fragments while they are newly or recently provided to the gel matrix and/or transwell insert, such as within about 1 hour to 24 hours, including about 2, 3, 4, 5, 6, 7, 8, 9, 10, 11, 12, 13, 14, 15, 16, 17, 18, 19, 20, 21, 22, and 23 hours. In further aspects, it may be desirable to allow the MV fragments a period to recover and/or reach a homeostatic state prior to administering the test compositions, such as from about 1 day to about 7 days, including about 2, 3, 4, 5, and 6 days. In further aspects, it may be desirable to allow the MV fragments to become stressed and/or starved in the serum-free medium prior to application of the test composition. In some aspects, it may be further desirable to allow for at least one change of the serum-free medium prior to application of the test composition. In additional aspects, it may be desirable to provide the test composition to the gel matrix prior to adding the MV fragments to the 3D in vitro culture. In some aspects, it may be desirable to provide the test composition more than once and/or in combination with another test composition or a composition known or presumably known for its effects on angiogenesis. In some aspects, it may be desirable to have a delayed and/or sustained release of the test composition. Such may be achieved by providing to the medium and/or the gel matrix a delayed or sustained release formulation, such as a biodegradable or bioerodible polymer-encapsulated formulation of the test composition.

In some aspects, the test composition is provided to the 3D gel culture for a desired period of time or as part of an overall time course evaluation examining the angiogenic effects after varying periods of exposure and/or elapsed time since contact. In some aspects, varying concentrations of the test composition may be applied either to each 3D in vitro cell culture or as part of a dose response study.

In some aspects, the test composition or compound is an agent of interest for its potential angiogenic inducing and/or stimulatory effects. In other aspects, the test composition or compound is an agent of interest for its potential angiogenic inhibitory effects. In some aspects, the agent of interest is a potential angiogenic agonist. In other aspects, the agent of interest is a potential angiogenic antagonist. In some aspects, the test composition or compound may be administered with one or more additional compounds, such as known agonists and/or antagonists, to assess the effect of the test composition on a known response, For example, administration or contacting the 3D in vitro culture with a known agonist and the test composition that yields further growth than the agonist alone may identify the test composition to be an additional agonist, a positive co-factor or a compound that adds a synergistic effect to the known agonist. Similarly if the same combination yields inhibited or reduced or arrested growth, the results help identify the test composition as an inert binding competitor or an antagonist.

In some aspects, the present disclosure concerns assessing the angiogenic effects that the test composition provides to the MV fragments in the 3D in vitro culture. In some aspects, the angiogenic effects can be determined by observing and/or measuring the MV length and/or changes in morphology and shape in response to the test composition. In some aspects, angiogenic changes can be determined by comparison to a control MV culture, such as one that is administered with no composition, a known agonist, a known antagonist, a placebo, or the vehicle in which the test composition is administered with. Such arrangements for comparison and/or validation of obtained results are known. In addition to serving as a flexible assay conducive to efficient evaluation of new drug targets of angiogenesis, the MV platform disclosed herein can be expanded to represent a variety of vascularized tissue assays such as tumors, wound healing, inflammation, etc. depending on the additional cell types added and the configuring of the tissue environment. Additionally, the presently disclosed platform can be used to screen the effects of any existing or emerging therapeutic.

In some aspects, the 3D in vitro culture includes intact native parent microvessels embedded in a matrix, such as a gel matrix. The 3D in vitro culture is exposed to dynamic conditions (i.e., low, medium, or high angiogenic conditions) and neovessel growth/sprouting is directly or indirectly quantified, using the techniques described herein. In some aspects, the angiogenic changes and/or growth can be determined visually. In other aspects, the angiogenic changes and/or growth can be determined and/or measured using measuring device and/or calculating devices to determine that amount of change and/or growth. In some aspects, the rate of change over a period of time may be observed and/or calculated. As set forth in the examples, one aspect of the present disclosure concerns artificial intelligence for microvessel quantification: In one aspect, assessments of angiogenesis involve measuring neovessel density in a manual fashion from fluorescence images. In another aspect, in order to obtain a more rapid, accurate assessment of angiogenesis, a Vascular Assessment and Measurement software (VAM) has been developed that utilizes artificial intelligence and machine learning (AI/ML) to identify and provide morphometric data from phase and fluorescence images of MV cultures. The VAM software is trained to recognize parent MV, neovessels, and non-MV artifacts. This analysis software functions coordinately with the Cytiva (formerly GE Healthcare) INCELL 6500 confocal scanning platform routinely used in the lab. In some aspects, visualization can be also achieved through antibody and/or fluorophore labeling.

In some aspects, the cells from the 3D culture can be assayed for varying levels of gene expression, enzymatic activity, and the like to assess for angiogenic effects, as well as through visualization and measurement of angiogenesis. Such may include immunohistochemistry, polymerase chain reactions, nucleotide isolation and/or blotting, protein purification and/or probing thereof, tandem mass spectrometry, phenotypic screening, sequencing, ELISA, electrophoresis, chromatography, flow cytometry, and combinations thereof. In some aspects, the 3D in vitro culture may be assessed with one or more traditional assay endpoints involving an MMP-14 ELISA, Alamar blue assay (as an angiogenesis biomarker, and readout metabolic activity, respectively), and combinations thereof. The model may comprise high content analysis (HCA) protocols to quantify angiogenic growth such as, for example, implementation of artificial intelligence and machine learning to identify and measure neovessel length density from automated confocal scans. In embodiments, the model provides a robust series of protocols and target readouts that enable throughput quantification of native angiogenic activity.

Accordingly, provided herein is a highly informative, robust, and proven 3D in vitro angiogenesis assay that captures the complexity of native angiogenesis in a format compatible with existing NCE discovery tools. In addition, the angiogenesis assay may validate a novel potential angiogenesis-related drug target, DYRK3, identified via a genomics screen using the assay.

In other aspects, as set forth herein, the present disclosure also concerns a model for assessing the potential of DYRK3 as a novel drug target for modulating angiogenesis. As the data herein demonstrate, RNA sequencing studies revealed differential expression of Dual Specificity Tyrosine Phosphorylation Regulated Kinase 3 (DYRK3) between microvessels with high and low angiogenic potentials. In some aspects, the angiogenesis model is employed to evaluate DYRK3 as a drug target. In other aspects, DYRK3 is selectively inhibited by performing a dose response of inhibitors. Gene networks downstream of DYRK3 activity may additionally be identified via RNA deep sequencing. Mechanisms of DYRK3 regulation of angiogenesis can be assessed by using techniques such as immunohistochemistry (IHC) and/or polymerase chain reaction (PCR) to determine which cell types from the microvessel wall express DYRK3, both relative to expression in parent and neovessels, and relative to different stages of angiogenesis and network formation. Assay methods disclosed herein are also useful in combination with the disclosed models.

The utility of this model is demonstrated by targeting the understudied protein Dual Specificity Tyrosine Phosphorylation Regulated Kinase 3 (DYRK3) as set forth in the Examples herein. While studies focusing on the function of DYRK3 have been limited, there are data to indicate it can promote cell survival [Guo, X., et al., DYRK1A and DYRK3 promote cell survival through phosphorylation and activation of SIRT1. J Biol Chem, 2010. 285(17): p. 13223-32], attenuate apoptosis [Li, K., et al., DYRK3 activation, engagement of protein kinase A/cAMP response element-binding protein, and modulation of progenitor cell survival. J Biol Chem, 2002. 277(49): p. 47052-60], and play a role in mTORC1 signaling [Wippich, F., et al., Dual specificity kinase DYRK3 couples stress granule condensation/dissolution to mTORC1 signaling. Cell, 2013. 152(4): p. 791-805]. Dysregulation of mTORC1 signaling has been linked to a number of diseases characterized by angiogenic pathologies, including cancer, diabetes, and others. In addition, new insights into the unique metabolism and cell survival of endothelial cells during angiogenesis further hints at a role for DYRK3 [Vandekeere, S., M. Dewerchin, and P. Carmeliet, Angiogenesis Revisited: An Overlooked Role of Endothelial Cell Metabolism in Vessel Sprouting. Microcirculation, 2015. 22(7): p. 509-17, Cantelmo, A. R., A. Brajic, and P. Carmeliet, Endothelial Metabolism Driving Angiogenesis: Emerging Concepts and Principles. Cancer J, 2015. 21(4): p. 244-9]. Due to the complexity of mTORC1 signaling, and because it affects so many processes, pharmaceuticals that target mTORC1 directly have been ineffective [Li, J., S. G. Kim, and J. Blenis, Rapamycin: one drug, many effects. Cell Metab, 2014. 19(3): p. 373-9]. However, if a more specific protein affecting mTORC1 signaling is targeted, such as DYRK3, it may be possible to create a more targeted and effective therapeutic response.

A first aspect of the present disclosure, either alone or in combination with any other aspects concerns a method for assessing angiogenic effects of a test composition, the method comprising: providing human microvessel fragments selected to correspond to a desired patient profile; embedding the human microvessel fragments in a gel matrix of a three dimensional (3D) in vitro culture; providing a serum free medium to the 3D in vitro culture comprising embedded human microvessel fragments; contacting the 3D in vitro culture comprising embedded human microvessel fragments with a test composition; and assessing the angiogenic effects of the test composition by measuring at least one angiogenic growth parameter of the 3D in vitro culture comprising embedded human microvessel fragments.

A second aspect of the present disclosure, either alone or in combination with any other aspects concerns the method of the first aspect, wherein the desired patient profile comprises a shared underlying condition or trait.

A third aspect of the present disclosure, either alone or in combination with any other aspects concerns the method of the first aspect wherein the desired patient profile comprises a heterogeneous selection of patients.

A fourth aspect of the present disclosure, either alone or in combination with any other aspects concerns the method of the first aspect wherein the gel matrix comprises collagen.

A fifth aspect of the present disclosure, either alone or in combination with any other aspects concerns the method of the first aspect wherein the serum free media is selected for low angiogenic growth conditions.

A sixth aspect of the present disclosure, either alone or in combination with any other aspects concerns the method of the first aspect wherein the serum free media is selected for medium angiogenic growth conditions.

A seventh aspect of the present disclosure, either alone or in combination with any other aspects concerns the method of the first aspect wherein the serum free media is selected for high angiogenic growth conditions.

An eighth aspect of the present disclosure, either alone or in combination with any other aspects concerns the method of the first aspect wherein angiogenic growth is quantified directly by measuring vessel length density of parent microvessels and neovessels to determine a neovessel:parent microvessel ratio.

A ninth aspect of the present disclosure, either alone or in combination with any other aspects concerns the method of the first aspect wherein angiogenic growth is quantified indirectly by Alamar Blue assay performed on a portion of culture media collected from the 3D in vitro culture.

A tenth aspect of the present disclosure, either alone or in combination with any other aspects concerns the method of the first aspect wherein angiogenic growth is quantified indirectly by MMP-14 assay performed on a lysate of the 3D in vitro culture.

An eleventh aspect of the present disclosure, either alone or in combination with any other aspects concerns the method of the first aspect further comprising suspending a permeable transwell over the gel matrix, wherein the permeable transwell comprises additional cells and further wherein the serum free media covers the additional cells in the permeable transwell.

A twelfth aspect of the present disclosure, either alone or in combination with any other aspects concerns the method of the eleventh aspect wherein the additional cells comprise macrophages.

A thirteenth aspect of the present disclosure, either alone or in combination with any other aspects concerns the method of the eleventh aspect wherein the additional cells are autologous to the human microvessels.

A fourteenth aspect of the present disclosure, either alone or in combination with any other aspects concerns the method of the eleventh aspect wherein the permeable transwell further comprises the gel matrix.

A fifteenth aspect of the present disclosure, either alone or in combination with any other aspects concerns the method of the first aspect wherein the test composition is determined to inhibit angiogenesis when quantified neovessel growth in the 3D in vitro culture contacted with the test composition is lower than the control value.

A sixteenth aspect of the present disclosure, either alone or in combination with any other aspects concerns the method of the first aspect wherein the test composition is determined to promote angiogenesis when quantified neovessel growth in the 3D in vitro culture contacted with the test composition is higher than the control value.

A seventeenth aspect of the present disclosure, either alone or in combination with any other aspects concerns the method of the first aspect further comprising comparing the angiogenic effects of the test composition of the 3D in vitro culture with angiogenic effects in a further 3D in vitro culture contacted with at least one compound known to influence angiogenesis.

An eighteenth aspect of the present disclosure, either alone or in combination with any other aspects concerns a three-dimensional (3D) in vitro culture comprising: a gel matrix comprised of a first tissue extract and collagen; a permeable transwell comprised of a second tissue extract; and a basal cell medium, wherein the permeable transwell is suspended over the gel matrix and further wherein the basal cell medium covers the second tissue extract.

A nineteenth aspect of the present disclosure, either alone or in combination with any other aspects concerns the 3D in vitro culture of the eighteenth aspect wherein the first tissue extract and the second tissue extract are each independently selected from the group consisting of human microvessels, macrophages, and mesenchymal stem cells, and further wherein the first tissue extract and the second tissue extract are not identical.

A twentieth aspect of the present disclosure, either alone or in combination with any other aspects concerns the 3D in vitro culture of the eighteenth aspect wherein the first tissue extract and the second tissue extract are autologous.

### EXAMPLES

3D In Vitro Human Angiogenesis Drug-Target Discovery Model

Currently, angiogenesis is assessed from isolated microvessels (MVs) by visualizing constructs under a microscope and assigning them a qualitative score between 0 and 5, with 0 for MV death, 0-1 for no growth, 2-3 for average/medium growth, and 4-5 for excessive growth. Alternatively, labor-intensive, computer-aided morphometry approaches are employed to extract quantitative length-density information from acquired images. While these approaches are useful, the MV system needs to be configured into a more quantitative throughput format to be most effective in drug-target discovery efforts. Provided herein is a robust, well characterized, throughput angiogenesis assay with multiple quantitative, functional readouts. Quantitative readouts of MV growth and angiogenesis biomarkers coupled with AI/ML-based morphometry measurements of neovessel growth are provided. This provides for accurate, consistent, and comprehensive measurements of angiogenesis.

### Experimental Design

For all assays, MVs isolated from human adipose tissue via a limited collagen digestion are embedded (100,000 MV/ml) in a 3 mg/ml collagen gel. This formulation is compatible with a variety of multi-well plates. Whenever possible, assessments are made daily over the course of 1 week, including image acquisition for HCA.

MV growth assessment: In one embodiment, the medium establishes medium or modest MV growth, allowing to assess the impact of a target involved in supporting or impeding MV growth. MV growth in the assays is validated over a range of angiogenesis levels (e.g. low, medium, and high) using defined media identified to establish consistent growth levels over a range of isolation lots (Table 2). MVs from a minimum of 3 different donors are used to account for donor-to-donor variation. Vessel length-density of parent and neovessels is measured at days 5 and 10 using the VAM software and INCELL confocal scanner and used to calculate neovessel:parent ratios (an indicator of sprouting potential). This results in a set of clear benchmarks for vessel density ratios associated with low, average, and high vessel growth.

Metabolic activity: It is known that endothelial cell metabolism changes during angiogenesis [Vandekeere, S., M. Dewerchin, and P. Carmeliet, Angiogenesis Revisited: An Overlooked Role of Endothelial Cell Metabolism in Vessel Sprouting. Microcirculation, 2015. 22(7): p. 509-17]. Thus, we use metabolic activity as an additional indicator of angiogenic growth. Alamar blue is commonly used as an indirect assessment of cellular proliferation and viability, by measuring metabolic activity. It has been used for many cell types including endothelial cells [Adya, R., et al., Visfatin induces human endothelial VEGF and MMP-2/9 production via MAPK and PI3K/Akt signalling pathways: novel insights into visfatin-induced angiogenesis. Cardiovasc Res, 2008. 78(2): p. 356-65, Guarnieri, D., et al., Effect of silica nanoparticles with variable size and surface functionalization on human endothelial cell viability and angiogenic activity. Journal of Nanoparticle Research, 2014. 16(2)], as it measures both oxidative and glycolytic metabolism [Abe, T., S. Takahashi, and Y. Fukuuchi, Reduction of Alamar Blue, a novel redox indicator, is dependent on both the glycolytic and oxidative metabolism of glucose in rat cultured neurons. Neuroscience Letters, 2002. 326: p. 179-182]. The Alamar Blue assay is performed using culture medium collected from the samples in culture for the previous experiment.

MMP-14 assay: MMP-14 is used as a molecular marker for quantifying neovessel growth. MV constructs are cultured for 0, 3, 7, 10, 14 and 21 days, then lysed and homogenized. The lysate is used for an MMP-14 ELISA. The day 0 serves as a standard readout for “no growth,” as there are no neovessels sprouting at this point. This allows to track MMP-14 levels in detail throughout all stages of angiogenesis. From this experiment, it can be determined if MMP-14 levels increase throughout all stages of sprouting growth, or if levels peak during sprouting, and decrease during later maturation stages.

Data

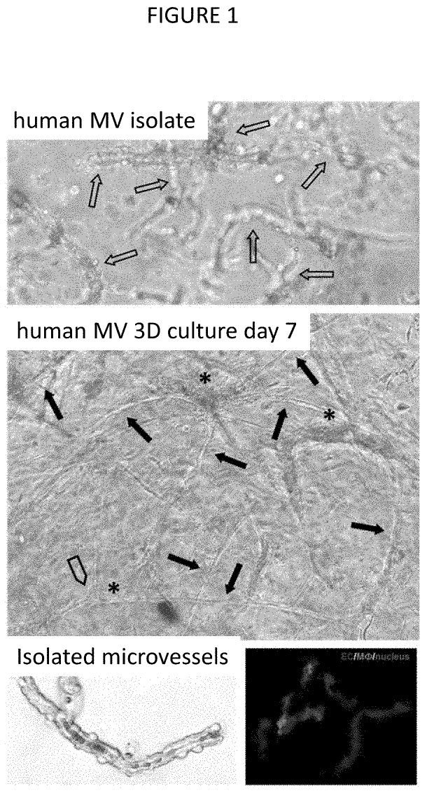

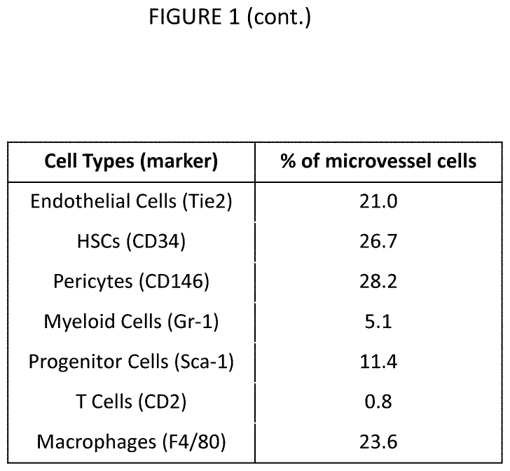

Microvessel Technology: Angiogenesis, vascular remodeling and vascular stability depend not only on the endothelial cell, but also proper vessel architecture, mature matrix elements, and a spectrum of perivascular cells [Schechner, J. S., et al., In vivo formation of complex microvessels lined by human endothelial cells in an immunodeficient mouse. Proc. Natl. Acad. Sci. U.S.A, 2000. 97(16): p. 9191-9196, Hellstrom, M., et al., Lack of pericytes leads to endothelial hyperplasia and abnormal vascular morphogenesis. Journal of Cell Biology, 2001. 153(3): p. 543-553, Caplan, A. I., All MSCs are pericytes? Cell Stem Cell, 2008. 3(3): p. 229-30]. With this in mind, an angiogenesis technology was developed utilizing freshly isolated microvessel fragments (FIG. 1), which contain all vascular cells types, maintained in the native microvessel structure [Hoying, J. B., C. A. Boswell, and S. K. Williams, Angiogenic potential of microvessel fragments established in three-dimensional collagen gels. In Vitro Cell Dev. Biol. Anim, 1996. 32(7): p. 409-419]. When the constructs are placed in 3D matrix cultures, the individual microvessels spontaneously sprout and grow, forming neovessels which will eventually fill the collagen gel (FIG. 1) [Hoying, J. B., C. A. Boswell, and S. K. Williams, Angiogenic potential of microvessel fragments established in three-dimensional collagen gels. In Vitro Cell Dev. Biol. Anim., 1996. 32(7): p. 409-419, Krishnan, L., et al., Interaction of angiogenic microvessels with the extracellular matrix. Am. J. Physiol Heart Circ. Physiol, 2007. 293(6): p. H3650-H3658]. When placed in a 3D matrix and implanted, the microvessels recapitulate angiogenesis/neovascularization and form stable, perfused, hierarchical microvascular networks [Shepherd, B. R., J. B. Hoying, and S. K. Williams, Microvascular transplantation after acute myocardial infarction. Tissue Eng, 2007. 13(12): p. 2871-9, Nunes, S. S., et al., Implanted microvessels progress through distinct neovascularization phenotypes. Microvasc. Res., 2010. 79(1): p. 10-20, Gruionu, G., et al., Encapsulation of ePTFE in prevascularized collagen leads to peri-implant vascularization with reduced inflammation. J Biomed. Mater. Res. A, 2010, Shepherd, B. R., et al., Rapid perfusion and network remodeling in a microvascular construct after implantation. Arterioscler. Thromb. Vasc. Biol., 2004. 24(5): p. 898-904]. Using rodent and human MVs, the progression of ischemic lesions following myocardial infarction are prevented and biomaterial biocompatibility in preclinical models is improved. Furthermore, a number of experimental investigations were performed investigating stromal cell and vascular precursor dynamics [Nunes, S. S., et al., Angiogenic potential of microvessel fragments is independent of the tissue of origin and can be influenced by the cellular composition of the implants. Microcirculation, 2010. 17(7): p. 557-67, Hiscox, A. M., et al., An islet-stabilizing implant constructed using a preformed vasculature. Tissue Eng Part A, 2008. 14(3): p. 433-40, Rhoads, R. P., et al., Satellite cell-mediated angiogenesis in vitro coincides with a functional hypoxia-inducible factor pathway. Am. J. Physiol Cell Physiol., 2009. 296(6): p. C1321-C1328], angiogenesis-tissue biomechanics [Krishnan, L., et al., Interaction of angiogenic microvessels with the extracellular matrix. Am. J. Physiol. Heart Circ. Physio.l, 2007. 293(6): p. H3650-H3658, Krishnan, L., et al., Effect of mechanical boundary conditions on orientation of angiogenic microvessels. Cardiovasc. Res., 2008. 78(2): p. 324-332, Krishnan, L., et al., Design and application of a test system for viscoelastic characterization of collagen gels. Tissue Eng., 2004. 10(1-2): p. 241-52], imaging modalities to assess neovascular behavior [Kirkpatrick, N. D., et al., Live imaging of collagen remodeling during angiogenesis. Am. J. Physiol. Heart Circ. Physiol., 2007. 292(6): p. H3198-H3206, Kirkpatrick, N. D., et al., In vitro model for endogenous optical signatures of collagen. J. Biomed. Opt., 2006. 11(5): p. 054021], and post-angiogenesis microvascular maturation and patterning [Chang, C. C., et al., Determinants of Microvascular Network Topologies in Implanted Neovasculatures. Arterioscler. Thromb. Vasc. Biol., 2011, Chang, C. C., et al., Angiogenesis in a microvascular construct for transplantation depends on the method of chamber circulation. Tissue Eng. Part A, 2010. 16(3): p. 795-805, Chang, C. C. and J. B. Hoying, Directed three-dimensional growth of microvascular cells and isolated microvessel fragments. Cell Transplantation, 2006. 15(6): p. 533-540]. This MV system can be applied as an in vitro experimental assay platform to evaluate angiogenic factors [Vartanian, K. B., et al., The non-proteolytically active thrombin peptide TP508 stimulates angiogenic sprouting. J. Cell Physiol., 2006. 206(1): p. 175-180], identify putative angiogenic agents [Carter, W. B. and M. D. Ward, Parathyroid-produced angiopoietin-2 modulates angiogenic response. Surgery, 2001. 130(6): p. 1019-1027, Carter, W. B., et al., Parathyroid-induced angiogenesis is VEGF-dependent. Surgery, 2000. 128(3): p. 458-64], evaluate microvascular instability [Carter, W. B., HER2 signaling—induced microvessel dismantling. Surgery, 2001. 130(2): p. 382-387], determine MMP-related angiogenic activity, and define matrix dynamics during angiogenesis [Krishnan, L., et al., Interaction of angiogenic microvessels with the extracellular matrix. Am. J. Physiol. Heart Circ. Physiol., 2007. 293(6): p. H3650-H3658, Krishnan, L., et al., Effect of mechanical boundary conditions on orientation of angiogenic microvessels. Cardiovasc. Res., 2008. 78(2): p. 324-332, Kirkpatrick, N. D., et al., Live imaging of collagen remodeling during angiogenesis. Am. J. Physiol. Heart Circ. Physiol., 2007. 292(6): p. H3198-H3206]. Recent work has focused on developing tissue vascularization applications with human-derived MVs. This past and ongoing work demonstrates the utility, flexibility, and versatility of the MVs. (Strobel et al., Stromal cells promote neovascular invasion across tissue interfaces. Frontiers Physiol. 2020, 11:1026, Strobel et al., Vascularized adipocyte organoid model using isolated human microvessel fragments. Biofabrication, 2021, 13(3): 035022).

Culture medium screening: Human MVs grow in a variety of serum free culture mediums to differing degrees (Table 1). MV growth outcome was qualitatively scored from 0-5 (with 5 being the highest). This spectrum of media types compatible with MV-based angiogenesis reflects the flexibility of the system and the potential of incorporating more fastidious parenchymal cell types into the assay.

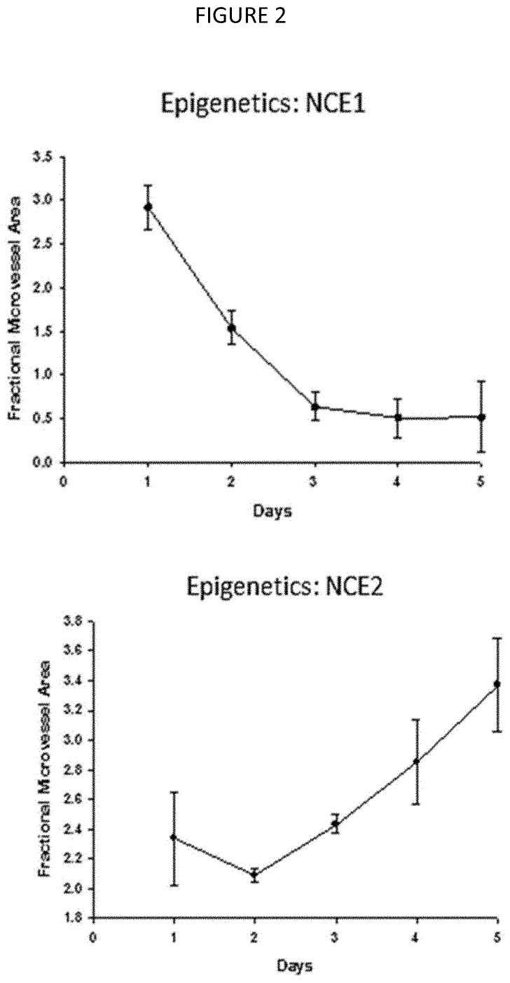

High Content Analysis of MV-based angiogenesis: A phenotypic screen of a library of 120 inhibitors of a variety of epigenetic targets (Selleckchem, Inc.) was performed on MV cultures for their effects on angiogenesis in a 96 well plate format. In the screen, angiogenesis was assayed under serum-free conditions that modestly promote angiogenesis (DMEM/F12+SATO+VEGF) (i.e. medium growth), enabling the identification of agents that either stimulate or enhance angiogenesis in a single screen. In the assay, neovessels sprout from the seeded, parent MV resulting in an increase in total vessel length over time (measured as fractional MV area to control for sampling variations). MV cultures were analyzed in the absence or presence of 10 μM of each inhibitor. Multiple relevant drug candidates were identified, two of which are shown in FIG. 2. The compound NCE1 inhibited angiogenesis, while NCE2 stimulated angiogenesis. This experiment also highlights the feasibility of using HCA with the MV system.

Artificial intelligence for microvessel quantification: In one aspect, assessments of angiogenesis involve measuring neovessel density in a manual fashion from fluorescence images. In another aspect, in order to obtain a more rapid, accurate assessment of angiogenesis, an application, BioSegment™, was developed that utilizes artificial intelligence and machine learning (AI/ML) to identify and provide morphometric data from phase and fluorescence images of MV cultures. The BioSegment™ software has been “trained” to recognize parent MV, neovessels, and non-MV artifacts. This analysis software functions coordinately with the Cytiva (formerly GE Healthcare) INCELL 6500 confocal scanning platform routinely used in the lab.

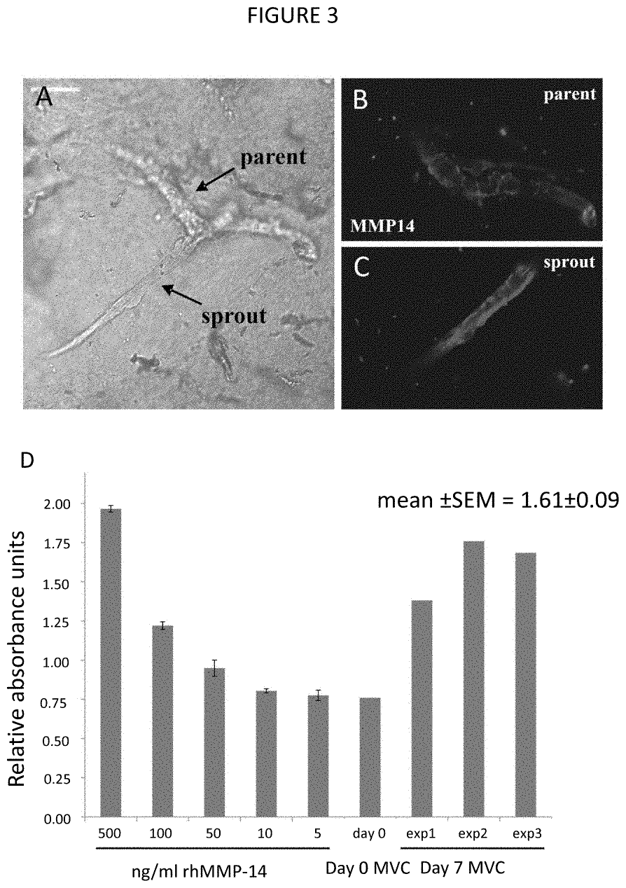

MMP-14 as a biomarker for neovessel growth: MMP-14 is known to play a role in angiogenesis [46, 47]. This led to testing for the presence of MMP-14 in the MV cultures, as a possible quantitative molecular indicator of angiogenesis. Using IHC, relatively specific expression of MMP-14 in growing neovessels was observed as compared to parent MVs (FIG. 3). With this in mind, an ELISA was developed for MMP-14 in MV cultures. A control day zero construct (no angiogenic sprouts) showed noise-floor levels of MMP-14, while 7-day old constructs (sprouting clearly visible) showed higher levels of MMP-14 (FIG. 3). This serves as a molecular readout of the assay.

Collectively, these studies show the development of an angiogenesis assay format that is validated via morphometric (high content analysis), molecular (MMP-14), and metabolic (Alamar blue) endpoints, and establishes benchmarks for high, medium, and low angiogenic growth for each of these endpoints. The BioSegment™ program provides a minimum of 90% accuracy compared to manual measurements. Optionally, the algorithm's accuracy is improved with inclusion of additional training data sets. Additionally, changes to its algorithms may be made as needed.

Donor Profile

A genomic screen of the MV cultures provided a potential advantage through the intrinsic heterogeneity in different lots of isolated MV, caused by donor to donor variability. While beneficial, this heterogeneity can also create variability in an assay. Selecting non-homogeneous donors or donors with a shared characteristic or trait can address the variability as well as identify compounds specific for a particular trait, disease, or shared characteristic found in subpopulations of patients.

Transwell Assays

Permeable transwell inserts can be used as an alternative format for screening microvessel behavior. Different types of cells, such as macrophages, mesenchymal stem cells, or others, can be seeded in a transwell insert, with or without a matrix (FIG. 7). This allows cells to secrete signaling molecules into the culture medium, exposing microvessels to those molecules, without coming in contact with the cells themselves. This can show the effect of cell-conditioned medium on microvessel growth. Alternatively, microvessels can be placed in the transwell, and cells in the main well, to evaluate the effect of microvessels on cellular behavior (FIG. 8).

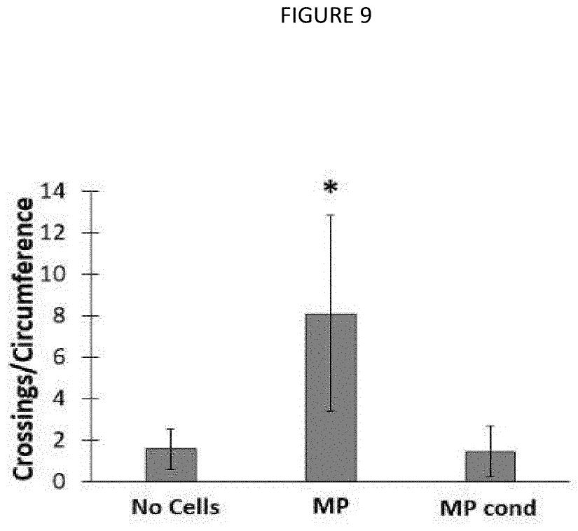

In FIG. 9, microvessels were cultured in a tissue interface model. Here, microvessels are in one compartment of collagen, and their ability to grow across a tissue interface into a secondary compartment is evaluated. Transwell inserts were utilized to determine the mechanisms that cause microvessels to cross an interface. Microvessels were plated in the tissue interface system, and either cultured alone, with macrophages (MP) mixed in the construct, or with macrophages in a transwell insert (MP cond). It was found that mixing MP with microvessels increased microvessel interface crossings, but MP in a transwell did not. This indicates that MP must be spatially near microvessels to influence crossing behavior, and conditioned medium is not sufficient.

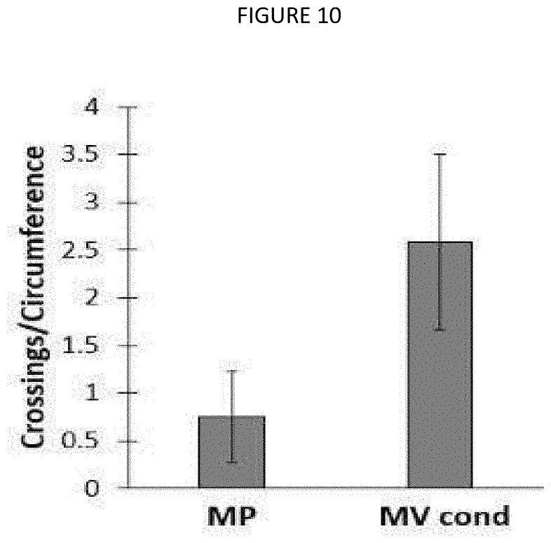

Next, the opposite experiment was performed, seeding MP in the interface model and MV in the transwell. Macrophages alone rarely crossed the interface. However, crossings were increased when microvessels were in the transwell above macrophages. This indicates that microvessels secrete signaling molecules that drive MP to cross the tissue interface (FIG. 10).

Validating the potential of DYRK3 as a novel drug target for modulating angiogenesis

DYRK3 is a member of the DYRK family of protein kinases (including DYRK1A,1B and DYRK2), which catalyze phosphorylation on serine, threonine, and tyrosine residues. The role of DYRK3 in cell biology and related processes is emerging [Bogacheva, O., et al., DYRK3 dual-specificity kinase attenuates erythropoiesis during anemia. J. Biol. Chem., 2008. 283(52): p. 36665-75, Guo, X., et al., DYRK1A and DYRK3 promote cell survival through phosphorylation and activation of SIRT1. J. Biol. Chem., 2010. 285(17): p. 13223-32]. The exact role of DYRK3 in angiogenesis remains unknown. Interestingly, DYRK1A positively regulates VEGF-dependent NFAT transcriptional responses in primary endothelial cells [Rozen, E. J., et al., DYRK1A Kinase Positively Regulates Angiogenic Responses in Endothelial Cells. Cell Rep., 2018. 23(6): p. 1867-1878], suggesting a possible link between this gene family and angiogenesis. Given DYRK3 promotes cell survival and DYRK3 activation can attenuate apoptosis [Li, K., et al., DYRK3 activation, engagement of protein kinase A/cAMP response element-binding protein, and modulation of progenitor cell survival. J. Biol. Chem., 2002. 277(49): p. 47052-60], we hypothesizzed that DYRK3 may be acting to stabilize the neovasculature during the dynamic activities of angiogenesis. Furthermore, DYRK3 mediates mTORC1 (mechanistic target of rapamycin) during cell stress [Wippich, F., et al., Dual specificity kinase DYRK3 couples stress granule condensation/dissolution to mTORC1 signaling. Cell, 2013. 152(4): p. 791-805], consistent with a possible role in vascular cell stability.

### Experimental Design

DYRK3 inhibition: Preliminary studies suggest that DYRK3 inhibition halts and potentially reverses angiogenesis. In some aspects, the scope of the preliminary experiment with the GSK626616 DYRK3 inhibitor is expanded to include dose responses and timing effects. Following initial sprouting of MVs in 96 well plates, 0.1, 1, and 10 μM doses of the inhibitor are each applied for 30 minutes, 3 hours, or 12 hours followed by washout. MVs are cultured for an additional 5 days, to observe changes in angiogenic growth. From this, a single dosing regimen is evaluated at day 0, 5, or 10 representing different phases of angiogenesis. These multiple time points allow for determination if the inhibitor is simply halting new neovessel growth, or additionally reversing network formation. MV length-density is quantified daily using the BioSegmentTMsoftware and compared to untreated controls. Culture medium harvested from all samples is used for an Alamar Blue assay to determine metabolic activity for each condition. Cultures are digested and used to measure MMP-14 levels via ELISA. These outcomes are compared to a standard angiogenesis sprouting assay [Sun, X.-T., et al., Angiogenic synergistic effect of basic fibroblast growth factor and vascular endothelial growth factor in an in vitro quantitative microcarrier-based three-dimensional fibrin angiogenesis system. World J. Gastroenterol., 2004. 10(17): p. 2524-2528] undergoing the same DYRK3 inhibition.

RNA sequencing: To evaluate signaling pathways downstream of DYRK3, evaluate gene expression using RNA sequencing is evaluated as before. Constructs are untreated or treated on day 5 with GSK626616 at concentrations and incubation times determined above, then flash frozen either immediately after treatment or after an additional 5 days of culture. RNA samples from these constructs are used in the transcriptome analysis. Genomic and principal component analysis are performed as previously described [Qiao, C., et al., Deep transcriptomic profiling reveals the similarity between endothelial cells cultured under static and oscillatory shear stress conditions. Physiol. Genomics, 2016. 48(9): p. 660-6] to identify potential pathways and gene clusters DYRK3 inhibition is affecting using Bioconductor freeware.

Assessment of DYRK3 location: Immunohistochemistry (IHC) for DYRK3 is performed on isolated MVs and cultured angiogenic constructs. DYRK3 is co-stained with markers for other cells, including CD11 for macrophages, UEA-1 lectin for endothelial cells, and smooth muscle alpha actin for smooth muscle cells and pericytes. MSC markers CD90 and CD105 are also stained, as MSCs can be more challenging to identify. This determines which of the many microvascular cell types present in the MV system are expressing DYRK3 and where in the growing neovessel DYRK3 is present (e.g. neovessel stalk, tip, or parent vessel).

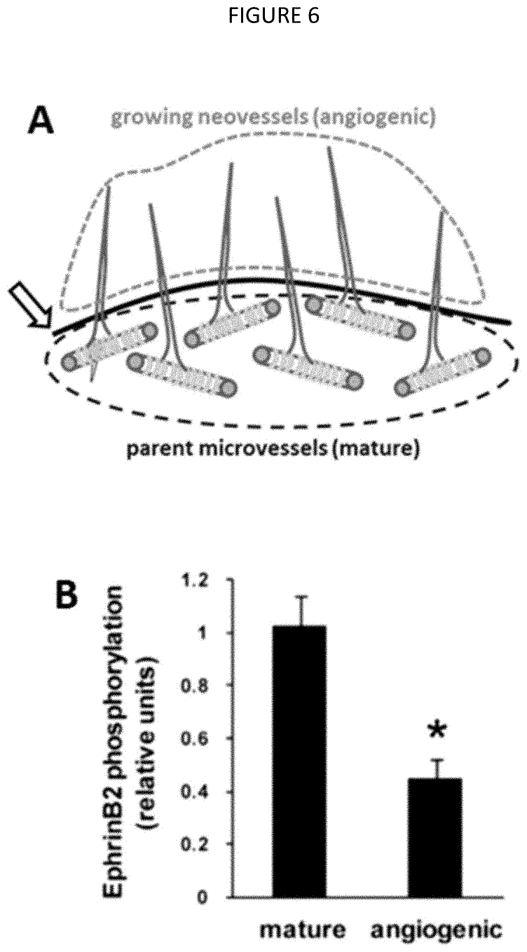

Time course of DYRK3 expression: Real-time PCR of DYRK3 transcripts in MV cultures is performed daily for 12 days to determine the time course of expression. To distinguish DYRK3 expression between actively growing neovessels and parent microvessels, the “core in field” model to allow for the harvesting of only neovessels that are actively growing (FIG. 6). In this model, parent microvessels are placed on one side of a matrix interface which is crossed by actively growing neovessels. Thus, the field is entirely neovessels, and the core is parent vessels and some neovessels. This shows how DYRK3 expression is changing in the parent vs neovessels over time.

DYRK3 activity: To obtain a functional measure of DYRK3 activity, changes in DYRK3 auto and substrate phosphorylation are quantified in the absence or presence of the DYRK3 inhibitor. MV constructs in the “core in field” format are treated with GSK626616 on day 5 of culture, at a concentration and incubation period determined in previous experiments. Constructs are lysed and homogenized either 20 mins after treatment or after 5 days of culture, in addition to 10-day untreated controls. Western blotting is performed on lysates of mature and angiogenic vessels, to quantify phosphorylated serine and threonine and also immunoprecipitated PRAS40, a known substrate of DYRK3 involved in mTORC1 signaling.

Data

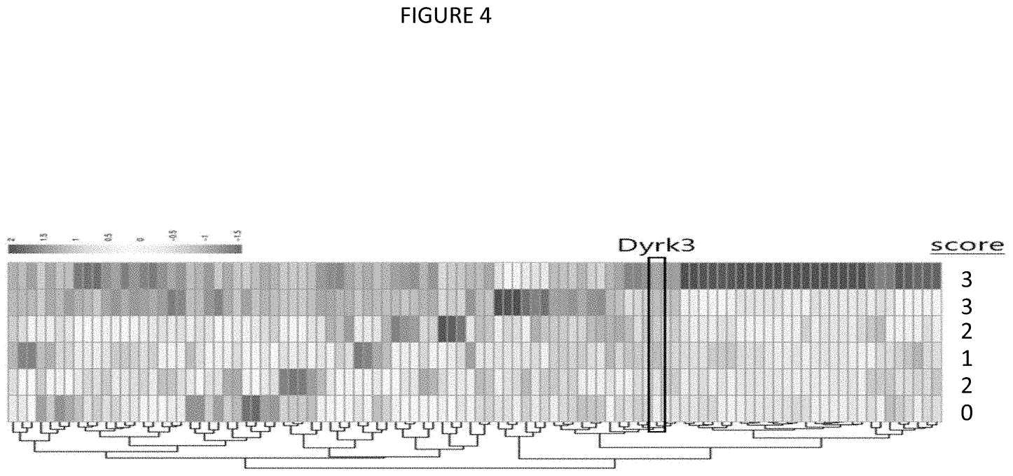

Deep sequencing screen for angiogenesis: As with most primary-sourced biologics, human MVs from different adipose donors can exhibit a range of angiogenic potentials. Leveraging this heterogeneity, it was reasoned that expression cohorts unique to each lot of MVs may reflect angiogenic potential transcriptomes, providing a means to identify possible novel drug targets. An initial screen involving deep sequencing of transcripts in MV isolates ranging in angiogenic potentials yielded 100 differentially expressed transcripts (FIG. 4). In a blinded analysis, those MV lots with good angiogenic potential (score of 3) segregated together and separately from MV lots with poor angiogenic potential (0-2). DYRK3 is one of the genes showing upregulation in high but not low angiogenic potential MV lots. Furthermore, DYRK3 is an interesting, potentially novel, kinase target identified in the Druggable Genome Initiative.

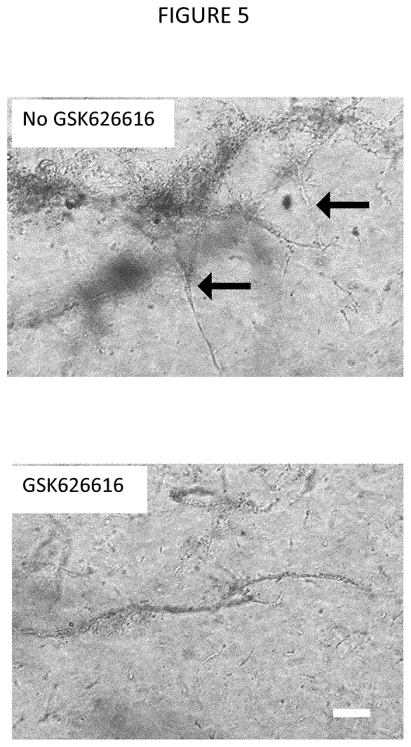

DYRK3 and angiogenesis: To further explore DYRK3 in this model, growing MVs are treated with the small molecule GSK626616, a known DYRK3 inhibitor. Cultures with growing neovessels (in RPMI with B27 and 50 ng/ml VEGF) were treated with 10 μM GSK626616 for 3 hours, and then returned to medium without inhibitor for an additional 6 days. Qualitatively, the inhibitor stopped neovessel growth and may have even caused neovessel regression, but did not impact parent MV, compared to the untreated controls (FIG. 5). The results suggest that DYRK3 influences angiogenesis.

The preliminary data suggests that DYRK3 inhibition halts neovessel growth and causes neovessel regression. Accordingly, the different measures of angiogenesis, including vessel density, MMP-14, Alamar blue, and phosphorylation, reflect this inhibition. Other inhibitors of DYRK3 may be explored, as needed.

Accordingly, provided herein is a robust, informative, 3D, in vitro angiogenesis assay that captures the complexity of native angiogenesis. This assay includes multiple quantitative and functional readouts, is simple and cost-effective to use, is conducive to moderate throughput and high content analysis and is compatible with existing drug target discovery tools. Additionally, the utility of the model is demonstrated herein, via evaluation of DYRK3, an understudied kinase, as a target for therapeutics targeting angiogenic dysregulation.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified. Methods of nucleotide amplification, cell transfection, and protein expression and purification are similarly within the level of skill in the art.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

We claim:

1. A method for assessing angiogenic effects of a test composition, the method comprising:
providing human microvessel fragments selected to correspond to a desired patient profile;
embedding the human microvessel fragments in a gel matrix of a three dimensional (3D) in vitro culture;
providing a serum free medium to the 3D in vitro culture comprising embedded human microvessel fragments;
contacting the 3D in vitro culture comprising embedded human microvessel fragments with a test composition; and
assessing the angiogenic effects of the test composition by measuring at least one angiogenic growth parameter of the 3D in vitro culture comprising embedded human microvessel fragments.

2. The method of claim 1, wherein the desired patient profile comprises a shared underlying condition or trait.

3. The method of claim 1, wherein the desired patient profile comprises a heterogeneous selection of patients.

4. The method of claim 1, wherein the gel matrix comprises collagen.

5. The method of claim 1, wherein the serum free media is selected for low angiogenic growth conditions.

6. The method of claim 1, wherein the serum free media is selected for medium angiogenic growth conditions.

7. The method of claim 1, wherein the serum free media is selected for high angiogenic growth conditions.

8. The method of claim 1, wherein angiogenic growth is quantified directly by measuring vessel length density of parent microvessels and neovessels to determine a neovessel:parent microvessel ratio.

9. The method of claim 1, wherein angiogenic growth is quantified indirectly by Alamar Blue assay performed on a portion of culture media collected from the 3D in vitro culture.

10. The method of claim 1, wherein angiogenic growth is quantified indirectly by MMP-14 assay performed on a lysate of the 3D in vitro culture.

11. The method of claim 1, further comprising suspending a permeable transwell over the gel matrix, wherein the permeable transwell comprises additional cells and further wherein the serum free media covers the additional cells in the permeable transwell.

12. The method of claim 11, wherein the additional cells comprise macrophages.

13. The method of claim 11, wherein the additional cells are autologous to the human microvessels.

14. The method of claim 11, wherein the permeable transwell further comprises the gel matrix.

15. The method of claim 1, wherein the test composition is determined to inhibit angiogenesis when quantified neovessel growth in the 3D in vitro culture contacted with the test composition is lower than the control value.

16. The method according to claim 1, wherein the test composition is determined to promote angiogenesis when quantified neovessel growth in the 3D in vitro culture contacted with the test composition is higher than the control value.

17. The method of claim 1, further comprising comparing the angiogenic effects of the test composition of the 3D in vitro culture with angiogenic effects in a further 3D in vitro culture contacted with at least one compound known to influence angiogenesis.

18. A three-dimensional (3D) in vitro culture comprising:
a gel matrix comprised of a first tissue extract and collagen;
a permeable transwell comprised of a second tissue extract; and
a basal cell medium,
wherein the permeable transwell is suspended over the gel matrix and further wherein the basal cell medium covers the second tissue extract.

19. The 3D in vitro culture of claim 18, wherein the first tissue extract and the second tissue extract are each independently selected from the group consisting of human microvessels, macrophages, and mesenchymal stem cells, and further wherein the first tissue extract and the second tissue extract are not identical.

20. The 3D in vitro culture of claim 18, wherein the first tissue extract and the second tissue extract are autologous.
